(19) United States
(12) Patent Application Publication
(10) Pub. No.: US 2023/0293070 A1
(43) Pub. Date: Sep. 21, 2023

(54) STRESS SCORE CALCULATION APPARATUS, METHOD, AND NON-TRANSITORY COMPUTER READABLE MEDIUM

(71) Applicant: KABUSHIKI KAISHA TOSHIBA, Tokyo (JP)

(72) Inventors: Mami SAITO, Fujisawa (JP); Takashi SUDO, Fuchu (JP); Yasuhiro KANISHIMA, Tokyo (JP); Izumi FUKUNAGA, Tokyo (JP)

(73) Assignee: KABUSHIKI KAISHA TOSHIBA, Tokyo (JP)

(21) Appl. No.: 17/940,028

(22) Filed: Sep. 8, 2022

(30) Foreign Application Priority Data
Mar. 17, 2022 (JP) 2022-042928

(51) Int. Cl. A61B 5/16; G06F 17/18

(52) U.S. Cl. CPC A61B 5/165; G06F 17/18

(57) ABSTRACT

According to one embodiment, a stress score calculation apparatus includes a memory and a processing circuit. The memory stores reference information which includes a reference value and/or a reference range pertaining to an action taken by a subject and/or a condition of an environment in which the subject is located that causes the subject an appropriate level of stress. The processing circuit acquires subject information which includes at least one of action information pertaining to the action taken by the subject and environment information pertaining to the environment in which the subject is located. The processing circuit calculates a score pertaining to a degree of stress experienced by the subject, based on a comparison between the subject information and the reference information.

### CROSS-REFERENCE TO RELATED APPLICATIONS

This application is based upon and claims the benefit of priority from Japanese Patent Application No. 2022-042928, filed Mar. 17, 2022, the entire contents of which are incorporated herein by reference.

### FIELD

Embodiments described herein relate generally to a stress score calculation apparatus, method, and non-transitory computer readable medium.

### BACKGROUND

In general, it is beneficial to evaluate the degree of stress experienced by a subject from the viewpoint of, for example, health risk management of the subject. Stress is classified as “acute stress”, which has a relatively short duration, and “chronic stress”, which has a relatively long duration. Acute stress is an instantaneous stress generated when a sudden change or a critical situation is encountered, and the degree of the acute stress is estimated using, for example, biometric information pertaining to bodily processes of the subject (examples: pulse wave, pulse rate, heart rate, amount of sweating, amount of urine, amount of saliva). However, chronic stress is stress accumulated over a long period of time, and the degree thereof is estimated using, for example, biomarkers (examples: blood, sweat, urine, and saliva) collected from a subject. Therefore, the degree of stress experienced by the subject can be evaluated based on both the degree of acute stress and the degree of chronic stress that are estimated for the subject.

However, there is no way of estimating the degree of chronic stress by using a simple, daily method. For example, there is a method of estimating the degree of chronic stress by using a wearable terminal to measure instantaneous stress before and after the subject sleeps. However, this method requires time and effort by the subject in wearing the terminal, which is not only inconvenient but also causes the subject unnecessary stress. As a result, it is not possible, using this method, to appropriately estimate and evaluate the degree of stress experienced by the subject.

### BRIEF DESCRIPTION OF THE DRAWINGS

FIG. 1 is a block diagram illustrating a functional configuration example of a stress score calculation apparatus according to a first embodiment.

FIG. 2 is a diagram illustrating an example of a reference information setting table according to the first embodiment.

FIG. 3 is a flowchart illustrating an example of an operation of the stress score calculation apparatus according to the first embodiment.

FIG. 4 is a diagram illustrating an example of a stress score conversion table according to the first embodiment.

FIG. 5 is a block diagram illustrating a functional configuration example of a stress score calculation apparatus according to a second embodiment.

FIG. 6 is a flowchart illustrating an example of an operation of the stress score calculation apparatus according to the second embodiment.

FIG. 7 is a block diagram illustrating a functional configuration example of a stress score calculation apparatus according to a third embodiment.

FIG. 8 is a flowchart illustrating an example of an operation of the stress score calculation apparatus according to the third embodiment.

FIG. 9 is a block diagram illustrating a functional configuration example of a stress score calculation apparatus according to a fourth embodiment.

FIG. 10 is a flowchart illustrating an example of an operation of the stress score calculation apparatus according to the fourth embodiment.

FIG. 11 is a diagram illustrating a display example of a stress score according to the fourth embodiment.

FIG. 12 is a block diagram illustrating a functional configuration example of a stress score calculation apparatus according to a fifth embodiment.

FIG. 13 is a flowchart illustrating an example of an operation of the stress score calculation apparatus according to the fifth embodiment.

FIG. 14 is a block diagram illustrating a hardware configuration example of the stress score calculation apparatuses according to the first to fifth embodiments.

### DETAILED DESCRIPTION

In general, according to one embodiment, a stress score calculation apparatus includes a memory and a processing circuit. The memory stores reference information which includes a reference value and/or a reference range pertaining to an action taken by a subject and/or a condition of an environment in which the subject is located that causes the subject an appropriate level of stress. The processing circuit acquires subject information which includes at least one of action information pertaining to the action taken by the subject and environment information pertaining to the environment in which the subject is located. The processing circuit calculates a score pertaining to a degree of stress experienced by the subject, based on a comparison between the subject information and the reference information.

Hereinafter, a stress score calculation apparatus, a method, and a non-transitory computer readable medium according to embodiments will be described with reference to the drawings. In the following embodiments, parts denoted by the same reference signs perform similar operations, and redundant descriptions will be omitted as appropriate.

### FIRST EMBODIMENT

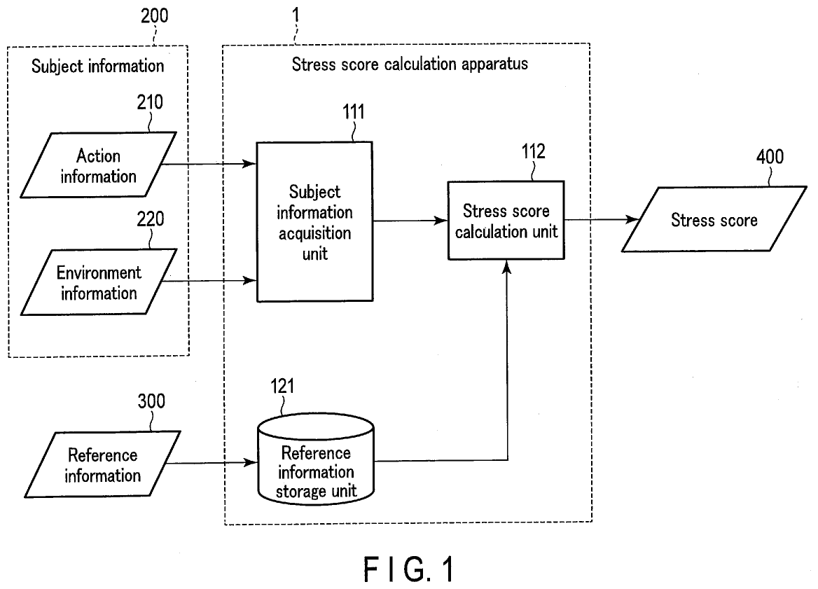

FIG. 1 is a block diagram illustrating a functional configuration example of a stress score calculation apparatus 1 according to a first embodiment. The stress score calculation apparatus 1 is an apparatus that calculates a score pertaining to the degree of stress (hereinafter also referred to as the “stress score 400”) experienced by the subject. Specifically, the stress score calculation apparatus 1 estimates and evaluates the degree of stress experienced by the subject, based on the appropriateness of the living environment of the subject (that is, whether or not the subject is experiencing appropriate stress). In the present specification, a human is assumed to be the “subject”, but the subject may be an animal other than a human (examples: dog, cat, horse, livestock). That is the stress score calculation apparatus 1 can be applied to the health risk management of humans and animals other than humans. The stress score calculation apparatus 1 includes, as components, a subject information acquisition unit 111, a reference information storage unit 121, and a stress score calculation unit 112.

The subject information acquisition unit 111 acquires various information pertaining to the subject (hereinafter also referred to as the “subject information 200”). For example, the subject information acquisition unit 111 acquires subject information 200 that includes at least one of action information 210 pertaining to an action taken by the subject and environment information 220 pertaining to an environment in which the subject is located. The action is, for example, resting, sunbathing, exercise, a meal, use of an electronic device, working, a meeting, a nap, a break, sleeping, weight measurement, a typing task, toilet, conversation, hydration, and/or respiration. The action information 210 is, for example, the content, amount, acceleration, time, time zone, and the count and/or frequency of the action taken by the subject. In addition, the action information 210 may be the facial color or facial expression, tone of voice, eye movement, weight, specific key typing count, posture, change in line of sight, number of health complaints, and/or mood of the subject. The action information 210 may also be a time, a time zone, or a length of time pertaining to sleep or work. The environment information 220 is, for example, the temperature, humidity, atmospheric pressure, and/or solar radiation amount of the environment in which the subject is located. In the present embodiment, the subject information acquisition unit ill acquires the action information 210 and the environment information 220 from the outside of the stress score calculation apparatus 1. In addition, the subject information acquisition unit ill transfers the acquired action information 210 and environment information 220 to the stress score calculation unit 112.

The reference information storage unit 121 stores reference information 300 which includes a reference value and/or a reference range pertaining to an action and/or an environment condition that causes the subject an appropriate level of stress. In the present embodiment, the reference information storage unit 121 acquires and stores the reference information 300 in advance from outside the stress score calculation apparatus 1. In addition, the reference information storage unit 121 transfers the stored reference information 300 to the stress score calculation unit 112.

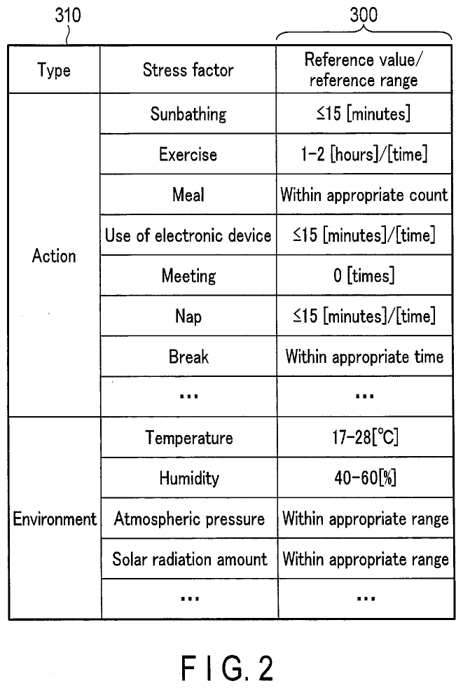

FIG. 2 is a diagram illustrating an example of a reference information setting table 310 according to the first embodiment. The reference information setting table 310 is a table in which a reference value and a reference range are set as the reference information 300 for each factor (stress factor) of an action or environment causing the subject to experience stress. According to another aspect, the reference information setting table 310 stores information pertaining to ideal regular life patterns that the subject should follow. The reference information setting table 310 also stores, for each stress factor, conditions that are necessary for the subject to experience an appropriate level of stress per unit period (example: one day, one week, one month, one year). Naturally, the reference information setting table 310 may also include, as stress factors, each of the items pertaining to the action information 210 and the environment information 220 described above. As a result, reference values and reference ranges pertaining to the stress factors may be set as the reference information 300.

In the reference information setting table 310, each stress factor caused by an action by the subject is associated with the reference value and the reference range for each stress factor. For example, the stress factor “sunbathing” is associated with a “value 15 [minutes]” indicating “15 minutes or less” as the reference range. Generally, sunbathing for about 15 minutes is ideal for the subject because same promotes the secretion of serotonin and stabilizes autonomic nerve balance. Based on such findings, “15 minutes or less” is set as the reference range for sunbathing.

Similarly, a value “1-2 [hours]/[time]” indicating “one hour to two hours per session” is associated with the stress factor “exercise” as the reference range. Generally, exercise for about one hour to two hours per session is ideal for the subject because the immune cells are activated so as to strengthen the immune system. Based on such findings, “1 hour to 2 hours per session” is set as the reference range for exercise. The stress factor “exercise” is not limited to these values, and a value of “exercise of a medium intensity for 20 minutes or more per day” may be associated with the stress factor “exercise” as the reference value. In addition, the reference information setting table 310 stores an appropriate time and value for each stress factor pertaining to actions by the subject (examples: resting, sunbathing, exercise, a meal, use of an electronic device, working, a meeting, a nap, a break, sleeping, weight measurement, a typing task, toilet, conversation, hydration, and respiration).

Furthermore, in the reference information setting table 310, each stress factor caused by an action by the subject is associated with the reference value and the reference range for each stress factor. For example, the stress factor “temperature” is associated with the reference range “17-28 [° C.]”. “Temperature” as defined here assumes the temperature (room temperature) of the room in which the subject is located, but may instead be the body temperature of the subject. Similarly, the stress factor “humidity” is associated with the reference range “40-60[%]”. In addition, the reference information setting table 310 stores an appropriate time and value for each stress factor (examples: atmospheric pressure, solar radiation amount, daylight hours, ultraviolet (UV) radiation) pertaining to the environment of the subject.

Note that the reference information 300 included in the reference information setting table 310 may also be manually set by the administrator of the stress score calculation apparatus 1 based on general knowledge. As a result, the administrator may appropriately edit and update the reference information 300 for each stress factor. Naturally, the stress score calculation apparatus 1 may autonomously acquire and update the reference information 300 from an external database or the like as appropriate. Hereinafter, it is assumed that the stress score calculation apparatus 1 pre-stores the reference information setting table 310 in the reference information storage unit 121.

The stress score calculation unit 112 calculates the stress score 400 pertaining to the degree of stress experienced by the subject, based on a comparison between the subject information 200 transferred from the subject information acquisition unit ill and the reference information 300 transferred from the reference information storage unit 121. Specifically, the stress score calculation unit 112 calculates the stress score 400 by adding a score indicating that the degree of stress experienced by the subject is appropriate in a case where the subject information 200 satisfies the reference information 300, and adding a score indicating that the degree of stress experienced by the subject is not appropriate in a case where the subject information 200 does not satisfy the reference information 300. The calculated stress score 400 is outputted to outside the stress score calculation apparatus 1. Hereinafter, the stress score 400 is assumed to be an index indicating the appropriateness of the degree of stress experienced by the subject. The stress score 400 is not limited thereto, and may be an index indicating the degree of stress itself that the subject experiences.

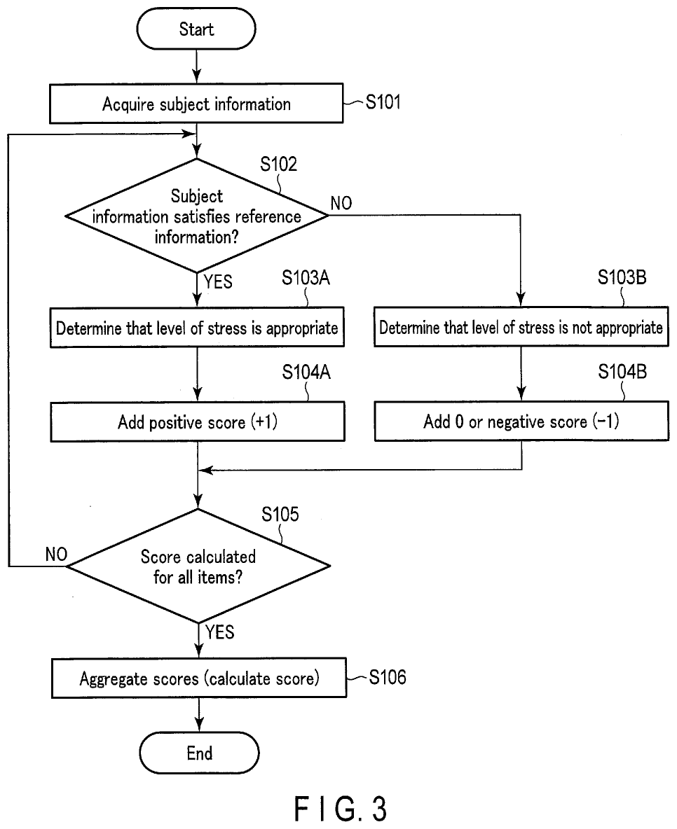

FIG. 3 is a flowchart illustrating an example of an operation of the stress score calculation apparatus 1 according to the first embodiment. This operation example may be started when the stress score calculation apparatus 1 receives a predetermined command from the administrator, or may be started without receiving the command. That is, the present operation example can be started in response to a request from the administrator, or can be automatically started periodically or with predetermined timing.

(Step S101) First, the stress score calculation apparatus 1 uses the subject information acquisition unit 111 to acquire subject information 200. For example, the stress score calculation apparatus 1 acquires, as the subject information 200, the action information 210 and the environment information 220 of the subject in a predetermined period (hereinafter also referred to as the “target period”) to be processed. The acquired action information 210 is used to estimate the degree of stress (action stress) experienced by the subject due to their own action during the target period. Meanwhile, the acquired environment information 220 is used to estimate the degree of stress (environment stress) experienced by the subject due to their surrounding environment during the target period. Note that the target period can be an arbitrary unit period (example: one day, one week, one month, one year).

The subject information 200 may be information collected by a sensor (examples: acceleration sensor, sound sensor, respiration sensor; temperature sensor, humidity sensor, atmospheric pressure sensor, illuminance sensor) or an interface (examples: GUI (Graphical User Interface), operation buttons) that is included in a portable terminal (examples: smartphone, wearable terminal, notebook PC, tablet terminal) owned by the subject. First, the action information 210 may be the intensity [METs], frequency, duration, or type of exercise performed by the subject collected by an acceleration sensor or the like, or may be the content, amount, time, time zone, count, or frequency of meals consumed by the subject. In addition, the action information 210 may be a conversation amount or an exhalation amount of the subject as collected by the sound sensor or the respiration sensor. Second, the environment information 220 may be a temporal change in temperature, humidity, atmospheric pressure, or solar radiation amount in the environment around the subject as collected by the temperature sensor, the humidity sensor, the atmospheric pressure sensor, or the illuminance sensor. That is, the action information 210 and the environment information 220 may be time-series data pertaining to the action and environment of the subject in the target period. Naturally, the action information 210 and the environment information 220 may be a set of numerical values including a plurality of continuous values or discrete values, or may be one specific numerical value or character string.

(Step S102) Next, the stress score calculation apparatus 1 uses the stress score calculation unit 112 to determine whether the subject information 200 satisfies the reference information 300. Specifically, the stress score calculation apparatus 1 compares each value of each item (stress factor) included in the subject information 200 with the reference information 300. At this time, for example, in a case where the values pertaining to the subject information 200 lie within the reference range of the reference information 300 or correspond to the reference values of the reference information 300, the stress score calculation apparatus 1 determines that the determination condition is satisfied. When the determination condition is satisfied (YES in step S102), the processing advances to steps S103A to S104A. However, when the determination condition is not satisfied (NO in step S102), the processing advances to steps S103B to S104B.

(Steps S103A to S104A) In this processing branch, the stress score calculation apparatus 1 uses the stress score calculation unit 112 to determine that the degree of stress experienced by the subject for the items to be processed in step S102 is appropriate, and adds a positive score (+1). That is, the positive score is an index indicating that the degree of stress experienced by the subject is appropriate.

(Steps S103B to S104B) In this processing branch, the stress score calculation apparatus 1 uses the stress score calculation unit 112 to determine that the degree of stress experienced by the subject for the items to be processed in step S102 is not appropriate, and adds 0 or a negative score (−1). That is, 0 or a negative score is an index indicating that the degree of stress experienced by the subject is not appropriate. Note that a negative score in comparison with the score “0” indicates that the degree of stress experienced by the subject is less appropriate.

(Step S105) Thereafter, the stress score calculation apparatus 1 uses the stress score calculation unit 112 to determine whether or not scores have been calculated for all items included in the subject information 200. When the processing has been completed for all the items (YES in step S105), the processing advances to step S106. However, when at least one unprocessed item exists (NO in step S105), the processing returns to step S102. That is, the processing according to steps S102 to S105 is executed iteratively until the processing is completed for all items.

(Step S106) Finally, the stress score calculation apparatus 1 uses the stress score calculation unit 112 to aggregate all the scores added in step S104A or S104B to calculate the stress score 400. Note that each of the scores may be regarded as the stress score 400, or a final score obtained by aggregating each of the scores may be regarded as the stress score 400. After execution of this step, the stress score calculation apparatus 1 ends the series of operations.

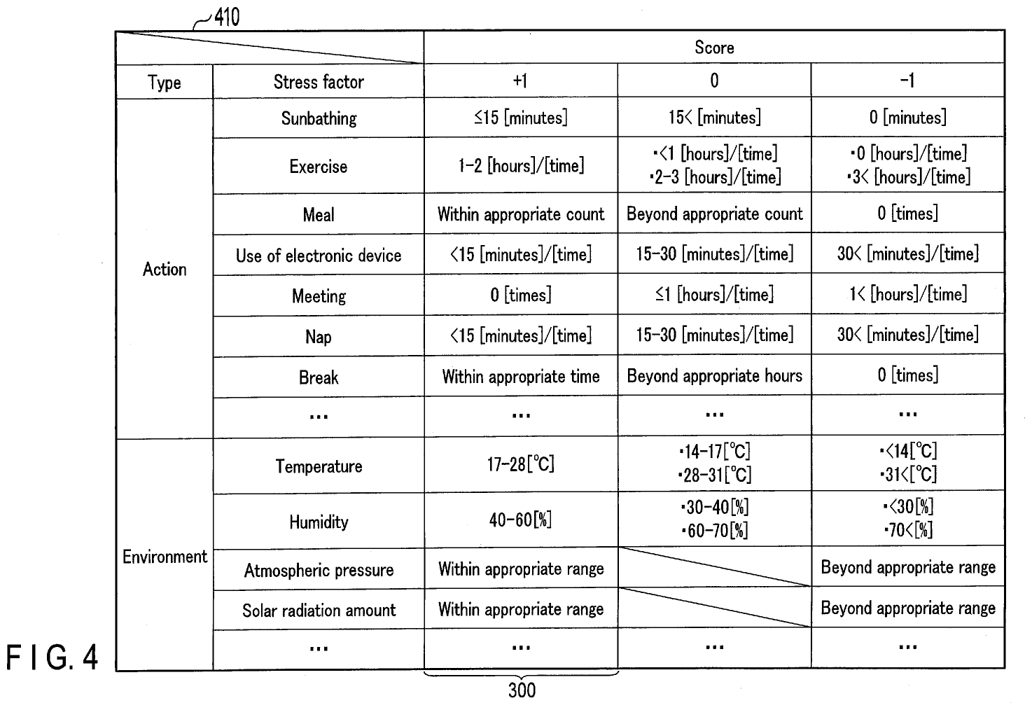

FIG. 4 is a diagram illustrating an example of a stress score conversion table 410 according to the first embodiment. The stress score conversion table 410 is a table in which conditions for converting values included in the subject information 200 into the stress score 400 are set. The stress score conversion table 410 may also be held and utilized by the stress score calculation unit 112. In the present embodiment, the stress score conversion table 410 converts values included in the subject information 200 into any of scores of three levels (+1, 0, −1). According to another aspect, the stress score conversion table 410 functions as a determination table referred to by the stress score calculation apparatus 1. Note that the score can be set in two positive and negative levels (+1, −1), or can be set in a plurality of levels which is four or more levels.

The score “+1” is set in the same range as the range included in the reference information 300 of the reference information setting table 310 described above. That is, when the reference information 300 in the reference information setting table 310 is updated, the reference information 300 in the stress score conversion table 410 is similarly updated. For example, in a case where the value of the subject information 200 lies within the range of the reference information 300, the value is converted into a score “+1”.

However, the scores “0” and “−1” are each set in a different range from the range included in the reference information 300 of the reference information setting table 310 described above. At this time, the range to which the score “0” is applied and the range to which the score “−1” is applied may be set so as not to overlap each other. For example, for the stress factor “sunbathing”, the range of the score “+1” is set to “15 [min]” indicating “15 minutes or less”, the range of the score “0” is set to “15<[min]” indicating “exceeding 15 minutes”, and the range of the score “−1” is set to “0 [min]”. Note that the range of each score may be manually set by the administrator of the stress score calculation apparatus 1, or may be automatically set by the stress score calculation apparatus 1 referring to an external database or the like.

Here, a case is assumed in which the stress score calculation apparatus 1 acquires observation data pertaining to “exercise” by the subject as the action information 210 and acquires observation data pertaining to the “solar radiation amount” of the subject as the environment information 220. In this case, the stress score calculation apparatus 1 uses the stress score conversion table 410 to determine which score each observation data item corresponds to. For example, in a case where the observation data for exercise is “1.5 [hours]/[time]”, because the observation data lies within the range of “1-2 [hours]/[time]”, a score “+1” is added for the observation data. However, when the observation data of the solar radiation amount is “within an appropriate range”, a score “+1” is added to the observation data. The stress score calculation apparatus 1 calculates “+2” as the stress score 400 by aggregating the added scores.

Note that in a case where the stress score calculation apparatus 1 acquires both the action information 210 and the environment information 220, the stress score calculation apparatus 1 may combine the information items with each other. For example, the stress score calculation apparatus 1 is capable of estimating that the subject performs the exercise outdoors by analyzing the correlation between the observation data for exercise and the observation data for the solar radiation amount. In general, outdoor exercise is ideal for the subject because same reduces the stress of the subject more effectively than indoor exercise. Therefore, the stress score calculation apparatus 1 may further add a score “+1” pertaining to “exercise outdoors” based on the estimated findings. As a result, the final stress score 400 is calculated as “+3” by adding a score “+1” based on outdoor exercise to the scores “+1” and “+1” obtained from the respective observation data. Thus, the stress score calculation apparatus 1 is capable of more accurately evaluating the degree of stress experienced by the subject by further adding a findings-based score estimated from a combination of information items to each score, based on each information item.

Conversely, in a case where both numerical values pertaining to the amount of exercise and the amount of solar radiation of the subject are high, the stress score calculation apparatus 1 is capable of estimating that the subject performs vigorous exercise outdoors. Generally, vigorous exercise causes the subject excessive stress in comparison with an appropriate level of exercise. The stress score calculation apparatus 1 may further add a score “−1” based on “vigorous exercise”, based on the estimated findings. Additionally, when the stress score calculation apparatus 1 combines various types of information, various synergistic effects are obtained.

The stress score calculation apparatus 1 according to the first embodiment has been described hereinabove. The stress score calculation apparatus 1 according to the first embodiment estimates and evaluates the degree of stress experienced by the subject, based on a comparison between the subject information 200 indicating the living environment of the subject which has actually been obtained and the reference information 300 indicating the ideal living environment, and converts the degree of stress into the stress score 400. By checking the stress score 400, the subject is able to grasp the degree of stress that they are experiencing due to disturbance of their living environment. Furthermore, according to the present method, because the subject does not need to wear a wearable terminal before or after sleeping, the subject is able to grasp the degree of stress they are experiencing without the hassle or burden of wearing the terminal while sleeping. Therefore, the stress score calculation apparatus 1 is capable of appropriately evaluating the degree of stress experienced by the subject.

Note that, as described above, the stress score calculation apparatus 1 calculates the stress score 400 based on a comparison between the action information 210 pertaining to the subject (examples: facial color or facial expression, tone of voice, eye movement, weight, specific key typing count, posture, change in line of sight, number of health complaints, and mood), and the reference information 300 corresponding to the action information 210. That is, the stress score calculation apparatus 1 performs various operations described below according to the type of information used to calculate the stress score 400.

First, the stress score calculation apparatus 1 uses the “facial color or expression” of the subject at a specific timing as the action information 210, and uses the “facial color or expression” of the subject in a normal state as the reference information 300. The stress score calculation apparatus 1 is capable of detecting a change in the complexion of the subject by comparing the two information items. For example, the stress score calculation apparatus 1 is capable of detecting the presence or absence of flushing in each part (examples: under the eyes, forehead, cheeks) of the face of the subject. In general, flushing of the face is a sign of tension, fatigue, or the like. From this perspective, the stress score calculation apparatus 1 enables the estimation accuracy of the chronic stress of the subject to be improved by using this information.

Secondly, the stress score calculation apparatus 1 uses the “tone of voice” of the subject at a specific timing as the action information 210, and uses the “tone of voice” of the subject in a normal state as the reference information 300. The stress score calculation apparatus 1 is capable of detecting a change in tone of the voice of the subject by comparing the two information items. For example, the stress score calculation apparatus 1 is capable of detecting whether or not the voice volume, intonation, and speech speed of the subject are lower than during a normal state. Generally, a reduction in a voice feature amount is a sign of tension, fatigue, or the like. From this perspective, the stress score calculation apparatus 1 enables the estimation accuracy of the chronic stress of the subject to be improved by using this information.

Thirdly, the stress score calculation apparatus 1 uses “eyeball movement” at a specific timing of the subject as the action information 210, and uses the “eyeball movement” in a normal state of the subject as the reference information 300. The stress score calculation apparatus 1 is capable of detecting a change in the amount of eye movement of the subject by comparing the two information items. For example, the stress score calculation apparatus 1 is capable of detecting whether or not the amount of eye movement of the subject increases in comparison with that during a normal state. In general, an increase in the amount of eye movement is a cause of asthenopia. In particular, when the subject is engaged in visual display terminal (VDT) work on a daily basis, the use of the foregoing information is beneficial. From this perspective, the stress score calculation apparatus 1 enables the estimation accuracy of the chronic stress of the subject to be improved by using this information.

Fourth, the stress score calculation apparatus 1 is capable of detecting whether or not the weight of the subject rapidly changes in a short period of time by comparing information pertaining to the “weight” at a specific timing and in a normal state, respectively, of the subject. Generally, a sudden change in body weight is attributed to stress. From this perspective, the stress score calculation apparatus 1 enables the estimation accuracy of the chronic stress of the subject to be improved by using this information.

Fifth, the stress score calculation apparatus 1 is capable of detecting a change in the number of inputs of a specific key (examples: delete key, backspace key) inputted by the subject by comparing information pertaining to “the specific key typing count” at a specific timing and in a normal state, respectively, of the subject. These illustrated keys are keys pertaining to deletion of inputted characters. Therefore, it can be said that an increase in the key input count is caused by a reduction in concentration due to stress. From this perspective, the stress score calculation apparatus 1 enables the estimation accuracy of the chronic stress of the subject to be improved by using this information.

Sixth, the stress score calculation apparatus 1 is capable of detecting a change in the posture of the subject by comparing information pertaining to the “posture” at a specific timing and in a normal state, respectively, of the subject. For example, the stress score calculation apparatus 1 is capable of detecting whether or not the subject engaged in VDT work has changed from an upright posture to a forward-bent posture (that is, the degree of concentration of the subject on the screen, and disturbance of the posture of the subject) with respect to the screen arranged in front of the subject. From this perspective, the stress score calculation apparatus 1 enables the estimation accuracy of the chronic stress of the subject to be improved by using this information.

Seventh, the stress score calculation apparatus 1 is capable of detecting a change in the amount of line-of-sight movement of the subject by comparing information pertaining to the “line-of-sight movement” at a specific timing and in a normal state, respectively, of the subject. For example, in a case where the amount of line-of-sight movement of the subject is relatively small, it is estimated that the subject gazes at one point in space for a long time. Generally, a reduction in the amount of line-of-sight movement is caused by chronic fatigue or a lack of sleep. From this perspective, the stress score calculation apparatus 1 enables the estimation accuracy of the chronic stress of the subject to be improved by using this information.

Eighth, the stress score calculation apparatus 1 is capable of detecting a change in the number of vague subjective symptoms, poor physical conditions, and instances of discomfort perceived by the subject by comparing information pertaining to the “number of health complaints” at a specific timing and in a normal state, respectively, of the subject. Generally, an increase in the number of health complaints is caused by stress. From this perspective, the stress score calculation apparatus 1 enables the estimation accuracy of the chronic stress of the subject to be improved by using this information.

### SECOND EMBODIMENT

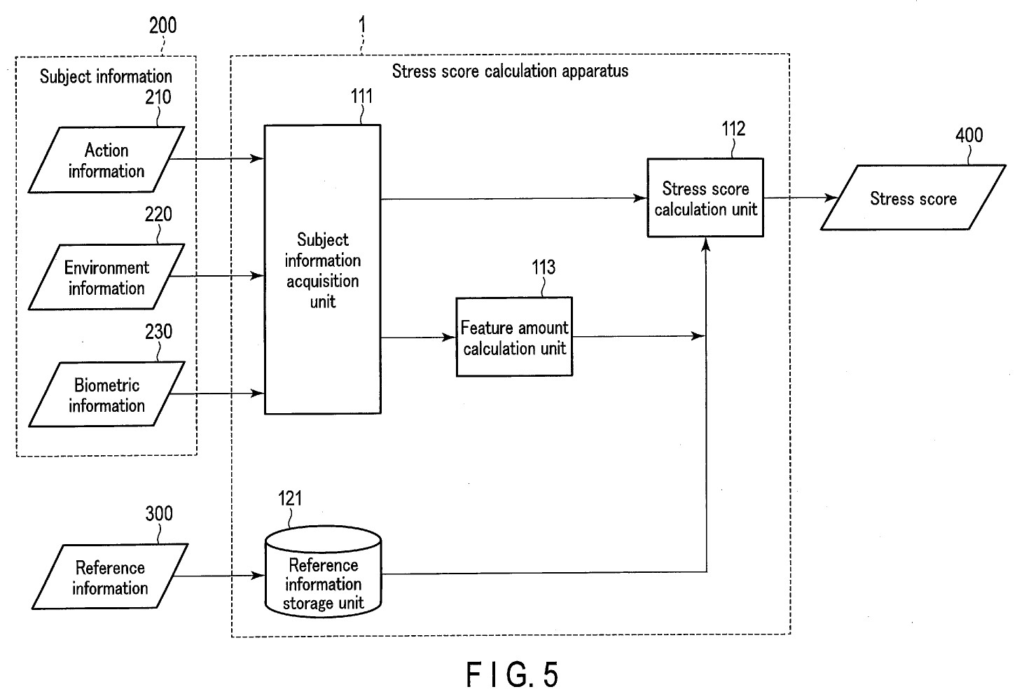

FIG. 5 is a block diagram illustrating a functional configuration example of a stress score calculation apparatus 1 according to a second embodiment. The stress score calculation apparatus 1 according to the second embodiment evaluates the degree of stress experienced by the subject, based on biometric information 230 on the subject in addition to the appropriateness of the living environment of the subject. The stress score calculation apparatus 1 includes, as components, a subject information acquisition unit 111, a reference information storage unit 121, a stress score calculation unit 112, and a feature amount calculation unit 113.

The subject information acquisition unit 111 further acquires biometric information 230 pertaining to bodily processes of the subject. The biometric information 230 is, for example, a pulse wave, a pulse rate, a heart rate, a perspiration amount, a urine amount, and/or a saliva amount. In the present embodiment, the subject information acquisition unit 111 acquires the biometric information 230 from outside the stress score calculation apparatus 1. Furthermore, the subject information acquisition unit 111 transfers the acquired biometric information 230 to the feature amount calculation unit 113.

The feature amount calculation unit 113 calculates a feature amount pertaining to a stress reaction of the subject, based on the biometric information 230 transferred from the subject information acquisition unit 111. The feature amount is, for example, an LF (Low Frequency)/HF (High Frequency) value, an LF value, an HF value, an ULF (Ultra Low Frequency) value, a VLF (Very Low Frequency) value, a total power value, an LF correction (LF norm) value, an HF correction (HF norm) value, an average HR (Heart Rate) value, an SDNN (Standard Deviation of the NN intervals: standard deviation of heartbeat intervals) value, an RMSSD (Root Mean Square of Successive Differences: Root Mean Square of Difference between consecutively adjacent heartbeat intervals) value, a CVRR (Coefficient of Variation of R-R Interval: coefficient of variation of heartbeat interval) value, an NN50 (total number of heartbeats in which a difference between adjacent heartbeat intervals exceeds 50 ms) value, a pNN50 (ratio of heartbeats in which a difference between adjacent heartbeat intervals exceeds 50 ms) value, an αLF value, a μPA value, a CV-PI value, a Lorenz plot, and/or a recurrent plot. That is, various autonomic nerve indices in the frequency domain or the time domain can be used as the feature amount. Note that the biometric information 230 may be directly used as the feature amount of a stress reaction.

In particular, a LF/HF value can be used as a stress index because same indicates the balance between the degree of sympathetic tone (LF value) and the degree of parasympathetic tone (HF value). For example, the LF/HF value is calculated every second from the wristband-type pulse wave sensor worn on the arm of the subject, whereby the time series data pertaining to the LF/HF value is obtained as the feature amount. In addition, the feature amount may be a function using various biomarkers (examples: urinary catecholamine, amylase, cortisol, chromogranin A) as references. The stress score calculation apparatus 1 enables the estimation accuracy of the stress of the subject to be improved by using a biomarker as a reference for long-term stress. Note that various known methods can be applied in the calculation of feature amounts. The feature amount calculation unit 113 transfers the calculated feature amount to the stress score calculation unit 112.

The stress score calculation unit 112 calculates the stress score 400 also based on the feature amount transferred from the feature amount calculation unit 113. Specifically, the stress score calculation unit 112 calculates the stress score 400 by adding a score indicating that the degree of stress experienced by the subject is appropriate in a case where the feature amount is less than or equal to a threshold value or lies within a predetermined range, and adding a score indicating that the degree of stress experienced by the subject is not appropriate in a case where the feature amount exceeds the threshold value or lies outside the predetermined range. The calculated stress score 400 is outputted to outside the stress score calculation apparatus 1. Note that, similarly to the first embodiment, the stress score calculation unit 112 calculates the stress score 400 by comparing the action information 210 and the environment information 220 in the subject information 200 with the reference information 300.

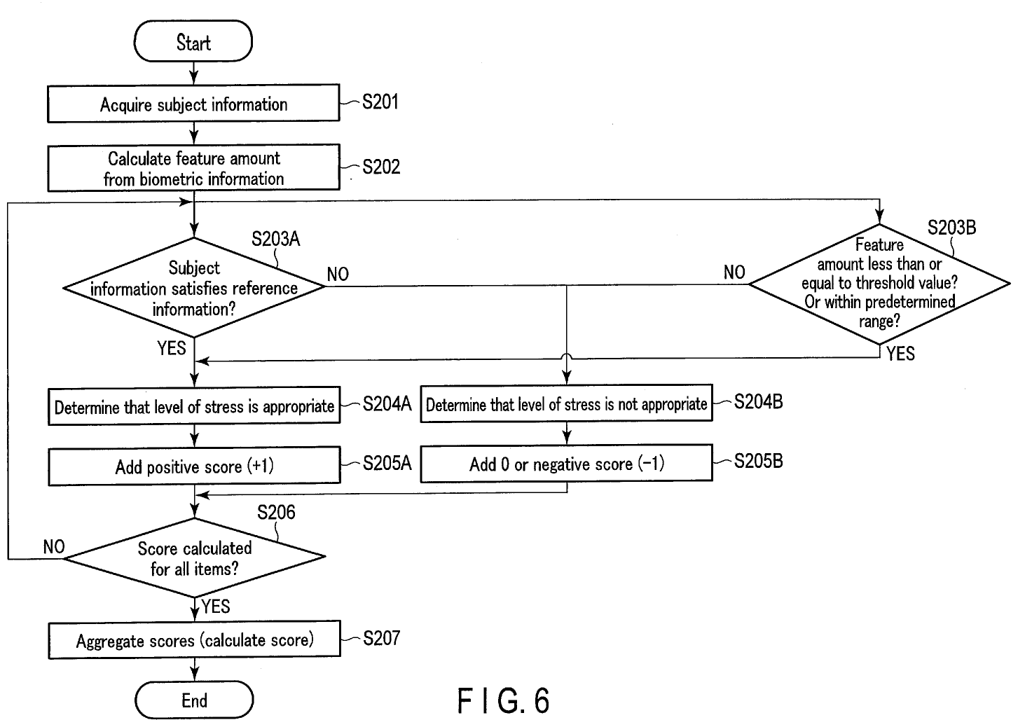

FIG. 6 is a flowchart illustrating an example of an operation of the stress score calculation apparatus 1 according to the second embodiment. In the present operation example, in addition to processing similar to that of the first embodiment, processing to calculate the stress score 400 based on the feature amount of the stress reaction is also performed.

(Step S201) First, the stress score calculation apparatus 1 uses the subject information acquisition unit 111 to acquire subject information 200. Specifically, the stress score calculation apparatus 1 acquires biometric information 230 in addition to the action information 210 and the environment information 220 of the subject in the target period, as the subject information 200. The acquired biometric information 230 is used to estimate the degree of acute stress experienced by the subject during the target period.

(Step S202) Next, the stress score calculation apparatus 1 uses the feature amount calculation unit 113 to calculate a feature amount from the biometric information 230. For example, when the biometric information 230 is time-series data pertaining to a pulse wave (heartbeat interval), the stress score calculation apparatus 1 calculates time-series data pertaining to an LF/HF value from the time-series data.

(Step S203A) Subsequently, the stress score calculation apparatus 1 uses the stress score calculation unit 112 to determine whether the subject information 200 (in particular, the action information 210 and the environment information 220) satisfies the reference information 300. Step S203A is similar to step S102. When the determination condition pertaining to step S203A is satisfied (YES in step S203A), the processing advances to step S204A. However, when the determination condition is not satisfied (NO in step S203A), the processing advances to steps S204B.

(Step S203B) However, the stress score calculation apparatus 1 uses the stress score calculation unit 112 to determine whether or not the feature amount calculated in step S202 is equal to or less than a threshold value or lies within a predetermined range. Here, for example, a case is assumed in which the feature amount is time-series data pertaining to LF/HF value. In a case where “0.8-2.0” is set as the appropriate range for the LF/HF value, the stress score calculation apparatus 1 determines whether the LF/HF value falls within the appropriate range over the time interval targeted by the time series data pertaining to the LF/HF value. The stress score calculation apparatus 1 determines that the above-described determination condition is satisfied when the LF/HF value is within the appropriate range. When the determination condition is satisfied (YES in step S203B), the processing advances to step S204A. However, when the determination condition is not satisfied (NO in step S203B), the processing advances to steps S204B.

Note that a threshold value or a predetermined range for the feature amount may be manually set by the administrator of the stress score calculation apparatus 1, or may be automatically set by the stress score calculation apparatus 1 referring to an external database or the like. Furthermore, in a case where there are a plurality of types of feature amounts, the determination processing according to step S203B can be executed for each of the plurality of types of feature amounts.

(Steps S204A to S205A) In this processing branch, the stress score calculation apparatus 1 uses the stress score calculation unit 112 to determine that the degree of stress experienced by the subject for the items to be processed in step S203A or S203B is appropriate, and adds a positive score (+1). Steps S204A to S205A are similar to steps S103A to S104A.

(Steps S204B to S205B) In this processing branch, the stress score calculation apparatus 1 uses the stress score calculation unit 112 to determine that the degree of stress experienced by the subject for the items to be processed in steps S203A or S203B is not appropriate, and adds 0 or a negative score (−1). Steps S204B to S205B are similar to steps S103B to S104B.

(Step S206) Thereafter, the stress score calculation apparatus 1 uses the stress score calculation unit 112 to determine whether or not scores have been calculated for all items included in the subject information 200 (that is, the action information 210, the environment information 220, and the biometric information 230). When the processing has been completed for all the items (YES in step S206), the processing advances to step S207. However, when at least one unprocessed item exists (NO in step S206), the processing returns to step S203A or S203B. That is, the processing according to step S203A or S203B to S206 is executed iteratively until the processing is completed for all items.

(Step S207) Finally, the stress score calculation apparatus 1 uses the stress score calculation unit 112 to aggregate all the scores added in step S205A or S205B to calculate the stress score 400. Step S207 is similar to step S106. After execution of this step, the stress score calculation apparatus 1 ends the series of operations.

The stress score calculation apparatus 1 according to the second embodiment has been described hereinabove. According to the stress score calculation apparatus 1 according to the second embodiment, advantageous effects similar to those of the first embodiment are obtained. Here, the stress score calculation apparatus 1 according to the second embodiment estimates and evaluates the degree of stress experienced by the subject, also based on the feature amount pertaining to the stress reaction of the subject obtained from the biometric information 230 of the subject, and converts the feature amount into the stress score 400. Therefore, the stress score calculation apparatus 1 according to the second embodiment is capable of more appropriately evaluating the degree of stress of the subject by further considering the index of the acute stress obtained from the biometric information 230 of the subject.

### THIRD EMBODIMENT

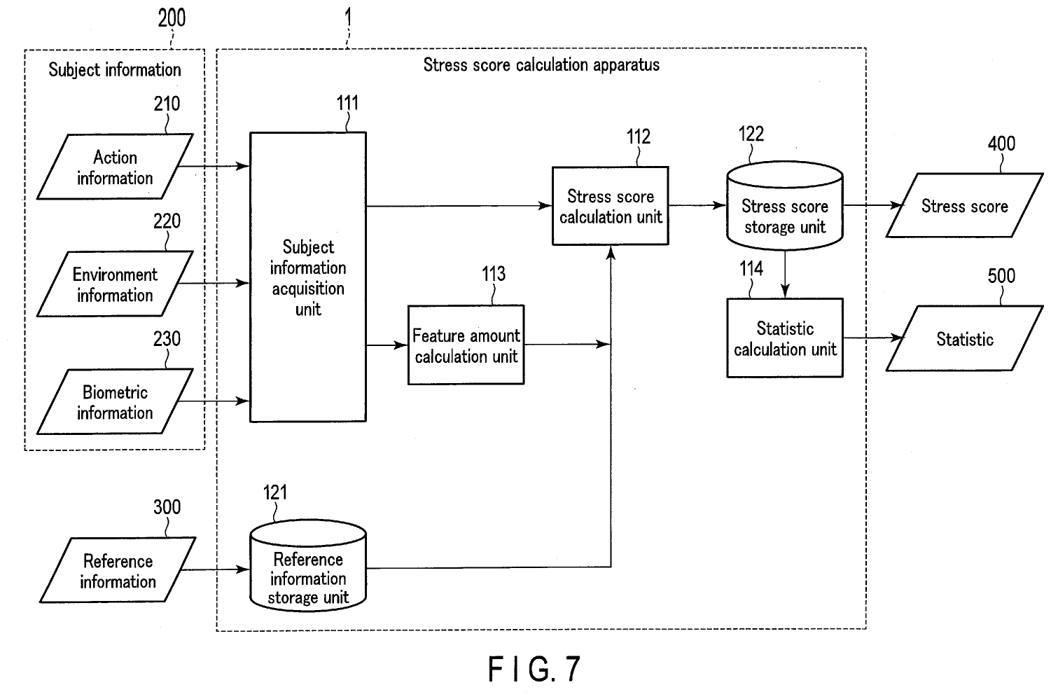

FIG. 7 is a block diagram illustrating a functional configuration example of a stress score calculation apparatus 1 according to a third embodiment. The stress score calculation apparatus 1 according to the third embodiment further includes a stress score storage unit 122 and a statistic calculation unit 114 in addition to the configurations included in the stress score calculation apparatus 1 according to the second embodiment.

The stress score storage unit 122 stores the calculated stress score 400 each time the stress score calculation unit 112 calculates the stress score 400. For example, when the stress score 400 is calculated every day, the stress score storage unit 122 stores and accumulates the stress score 400 every day. As a result, the stress score calculation apparatus 1 is capable of performing various statistical analyses by using the stored stress scores 400 for a plurality of days.

Naturally, the stress score storage unit 122 may store the stress scores 400 separately for each stress factor of the subject. In addition, the stress score storage unit 122 may store the stress score 400 over a certain period and then automatically delete the stored stress score 400. Further, the reference information storage unit 121 may further store the stress score 400 in addition to the reference information 300. The stored stress score 400 is outputted to outside the stress score calculation apparatus 1.

The statistic calculation unit 114 calculates a statistic 500 pertaining to each stress score 400 stored in the stress score storage unit 122. The statistic 500 is, for example, an average value, a total value, a minimum value, a maximum value, a median value, a deviation value, a mode value, a range, a variance, a standard deviation, a skewness, and/or kurtosis. For example, the statistic calculation unit 114 calculates, as the statistic 500, the stress scare 400 (baseline score) that is the average value per day over a one-week period, based on one-week stress scores 400. As a result, the statistic calculation unit 114 may calculate, as the statistic 500, the difference between the stress score 400 for each day included in the one-week stress scores 400 and the baseline score (the score change amount for each day; the deviation value). The calculated statistic 500 is outputted to outside the stress score calculation apparatus 1.

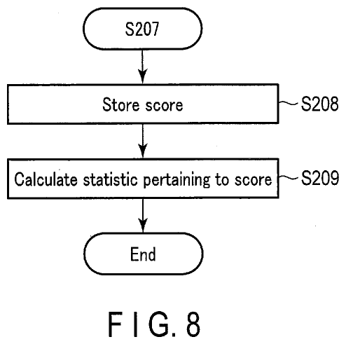

FIG. 8 is a flowchart illustrating an example of an operation of the stress score calculation apparatus 1 according to the third embodiment. The stress score calculation apparatus 1 according to the third embodiment executes the same operations (steps S201 to S207) as those of the second embodiment, and then further executes steps S208 and S209.

(Step S208) Here, the stress score calculation apparatus 1 stores the stress score 400 calculated in the immediately preceding step S207.

(Step S209) Finally, the stress score calculation apparatus 1 calculates the statistic 500 pertaining to the stress score 400. After execution of this step, the stress score calculation apparatus 1 ends the series of operations.

The stress score calculation apparatus 1 according to the third embodiment has been described hereinabove. With the stress score calculation apparatus 1 according to the third embodiment, advantageous effects similar to those of the first embodiment and the second embodiment are obtained. In addition, the stress score calculation apparatus 1 according to the third embodiment stores a plurality of stress scores 400, and further calculates various statistics 500 by using the plurality of stored stress scores 400. By checking the plurality of stress scores 400 and the statistics 500, the subject is able to easily understand a temporal change or the like in the degree of stress experienced by the subject. In particular, the subject is able to understand a cause of stress, an action that is effective in reducing stress, and so forth.

### FOURTH EMBODIMENT

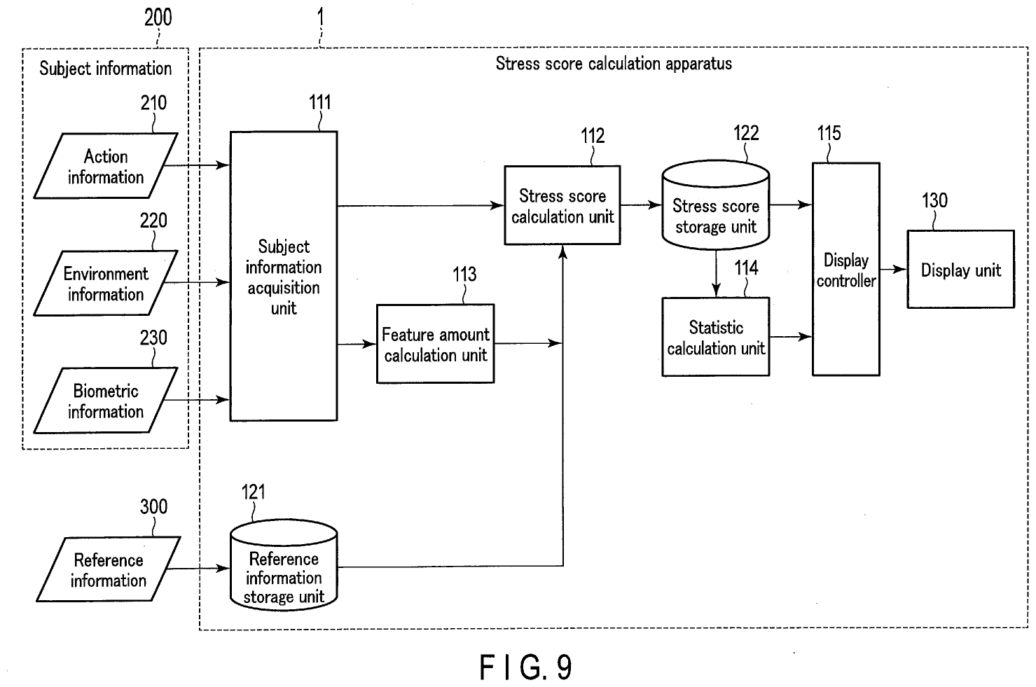

FIG. 9 is a block diagram illustrating a functional configuration example of a stress score calculation apparatus 1 according to a fourth embodiment. The stress score calculation apparatus 1 according to the fourth embodiment further includes a display controller 115 and a display unit 130 in addition to the configurations included in the stress score calculation apparatus 1 according to the third embodiment.

The display controller 115 performs control to cause the display unit 130 to display each stress score 400 stored in the stress score storage unit 122 and/or the statistic 500 calculated by the statistic calculation unit 114. For example, the display controller 115 performs control to cause the display unit 130 to display the stress scores 400 in a first predetermined period and the average value and/or the total value of the stress scores 400, and the stress scores 400 in a second predetermined period included in the first predetermined period and the average value and/or the total value of the stress scores 400. According to such display control, for example, the subject is able to compare data on a specific day with data (trend) in the entire period including the specific day, and qualitatively or quantitatively evaluate the significance of the former data. Note that the display controller 115 may generate image data pertaining to a display image for displaying the stress scores 400 and the statistics 500 in a predetermined mode, and transfer the generated image data to the display unit 130. In this case, the display unit 130 displays a display image based on the transferred image data. Incidentally, the display unit 130 may or may not be included in the stress score calculation apparatus 1.

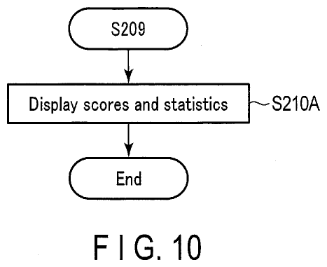

FIG. 10 is a flowchart illustrating an example of an operation of the stress score calculation apparatus 1 according to the fourth embodiment. The stress score calculation apparatus 1 according to the fourth embodiment executes the same operations (steps S201 to S209) as those of the third embodiment, and then further executes step S210A.

(Step S210A) Here, the stress score calculation apparatus 1 uses the display controller 115 and the display unit 130 to display the stress scores 400 and the statistics 500. After execution of this step, the stress score calculation apparatus 1 ends the series of operations.

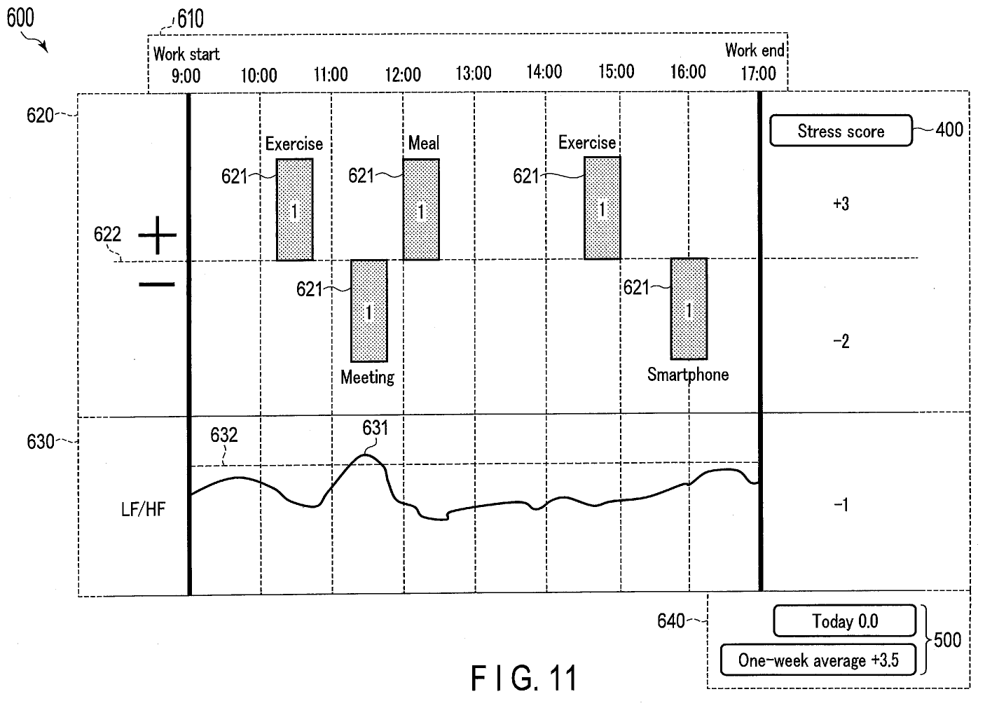

FIG. 11 is a diagram illustrating a display example of a stress score 400 according to the fourth embodiment. Here, a display image 600 that displays the stress score 400 and the statistics 500 pertaining to the subject in a predetermined mode is illustrated. The display image 600 can be displayed on the display unit 130. The display image 600 includes an area 610 indicating a period (target period) in which the stress score 400 is to be calculated, an area 620 indicating the stress score 400 due to chronic stress in the target period, an area 630 indicating the stress score 400 due to acute stress in the target period, and an area 640 indicating the statistics 500 calculated from the stress score 400 in the target period.

In the area 610, times from “work start: 9:00” to “work end: 17:00” are displayed as an example of the target period. Specifically, the eight hours from the work start time until the work end time are divided and displayed on an hour by hour basis.

Area 620 illustrates a history of increases/decreases in each stress score 400 corresponding to each action taken by the subject in the target period. Each stress score 400 is indicated by a band 621 which extends upward from a reference line 622 when the score is positive (+). Conversely, each stress score 400 is indicated by a band 621 which extends downward from reference line 622 when the score is negative (−). The vertical width of the band 621 is determined according to the magnitude of the score indicated by the band 621. However, the horizontal width of the band 621 is determined according to the continuous duration of the action indicated by the band 621. In this example, the vertical width and the horizontal width of each of the five bands 621 illustrated in the area 620 are the same. Note that the number of bands 621 indicates that the score corresponds to 1 point (+1 or −1).

For example, focusing on the band 621 in the time zone of “10:00-11:00”, it is understood that the score pertaining the “exercise” performed by the subject is “+1”. That is, the subject experiences an appropriate level of stress due to the exercise. Because there are a total of three bands 621 present above the reference line 622, “+3” is calculated as the total value of the positive stress scores 400 from these bands 621. Meanwhile, focusing on the band 621 in the time zone of “11:00-12:00”, it is understood that the score pertaining to the “meeting” which the subject attends is “−1”. That is, the subject experiences an inappropriate level of stress due to the meeting. Because there are a total of two bands 621 present below the reference line 622, “−2” is calculated as the total value of the negative stress scores 400 from these bands 621.

Area 630 displays a graph 631 indicating a temporal change pertaining to the LF/HF value of the subject in the target period and a reference line 632 for calculating the stress score 400 from the graph 631. Here, it is assumed that the higher the value of the graph 631, the larger the LF/HF value is. For example, when the reference line 632 indicates an upper limit of “2.0” of a proper value for the LF/HF value, it is understood that the value of the graph 631 exceeds “2.0” in the time zone of “11:00-12:00”. That is, the subject receives acute stress, which is inappropriate, in the time zone. The stress score calculation apparatus 1 calculates “1 time” as the number of times the value of the graph 631 exceeds the reference line 632. As a result, “−1” is calculated as the stress score 400 due to acute stress in the target period.

In the area 640, the total value of the stress scores 400 indicated in the area 620 and the area 630 is indicated as the statistic 500. Specifically, the stress score calculation apparatus 1 calculates the score “0.0” by summing the scores “+3” and “−2” in the area 620 and the score “−1” in the area 630. That is, the score “0.0” is the total value of the degrees of stress experienced by the subject in the target period indicated in the area 610. Furthermore, a score “+3.5”, which is the average value in a one-week period is displayed below the score “0.0”. By comparing the total value “0.0” of today's scores with the weekly average “+3.5”, the subject is able to evaluate the degree of stress they experienced on a specific day (today) in comparison with the entire period (one week).

The stress score calculation apparatus 1 according to the fourth embodiment has been described hereinabove. According to the stress score calculation apparatus 1 according to the fourth embodiment, advantageous effects similar to those of the first embodiment, the second embodiment, and the third embodiment are obtained. In addition, the stress score calculation apparatus 1 according to the fourth embodiment represents the stress score 400 and the statistics 500 using the display mode illustrated in the display image 600. By checking the display image 600 displayed on the display unit 130, the subject is able to intuitively and easily understand a temporal change or the like in the degree of stress they are experiencing.

### FIFTH EMBODIMENT

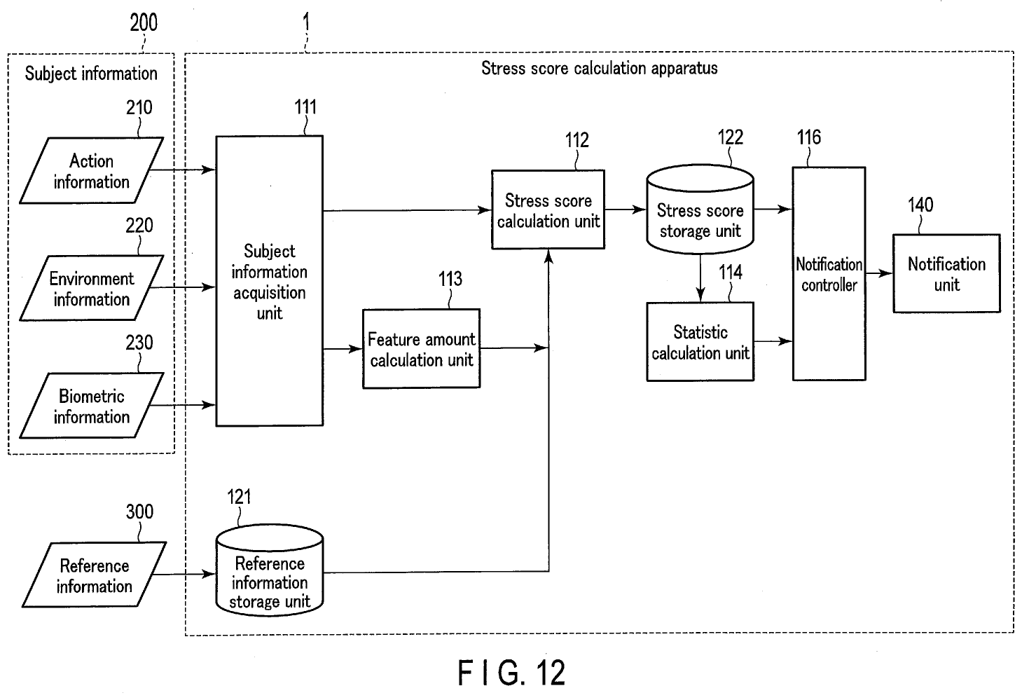

FIG. 12 is a block diagram illustrating a functional configuration example of a stress score calculation apparatus 1 according to a fifth embodiment. The stress score calculation apparatus 1 according to the fifth embodiment further includes a notification controller 116 and a notification unit 140 in addition to the configurations included in the stress score calculation apparatus 1 according to the third embodiment. In other words, the stress score calculation apparatus 1 according to the fifth embodiment is obtained by replacing the display controller 115 according to the fourth embodiment with the notification controller 116 and replacing the display unit 130 with the notification unit 140.

The notification controller 116 performs control to cause the notification unit 140 to issue a notification when each stress score 400 stored in the stress score storage unit 122 and/or the statistic 500 calculated by the statistic calculation unit 114 satisfies a predetermined criterion. Note that the predetermined criterion may be manually set by the administrator of the stress score calculation apparatus 1, or may be automatically set by the stress score calculation apparatus 1 referring to an external database or the like. However, an alert can be issued to the subject by a warning sound such as a beep sound. In addition, the alert may be a warning window displayed on the display unit 130. Incidentally, the notification unit 140 may or may not be included in the stress score calculation apparatus 1.

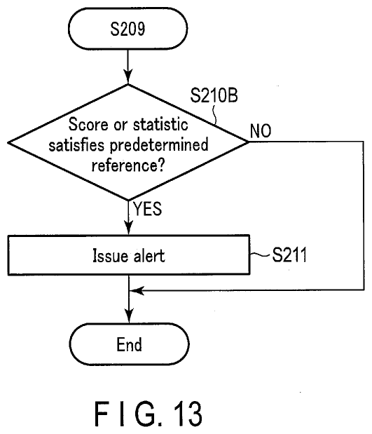

FIG. 13 is a flowchart illustrating an example of an operation of the stress score calculation apparatus 1 according to the fifth embodiment. The stress score calculation apparatus 1 according to the fifth embodiment executes the same operations (steps S201 to S209) as those of the third embodiment, and then further executes step S210B.

(Step S210B) Here, the stress score calculation apparatus 1 determines whether or not the stress score 400 or the statistic 500 satisfies a predetermined criterion. The predetermined criterion is, for example, whether or not the total value of the stress scores 400 of all stress factors is a negative value for N (N: natural number) consecutive days. Alternatively, the predetermined criterion is whether the total value of the stress scores 400 pertaining to a specific stress factor is a negative value for N consecutive days. The fact that the negative value of the stress score 400 is continuously calculated means that the subject continuously experiences unfavorable stress. Therefore, in such a case, it is desirable to notify and remind the subject of this fact. When the determination condition is satisfied (YES in step S210B), the processing advances to step S211. However, when the determination condition is not satisfied (NO in step S210B), the processing ends.

(Step S211) In this processing branch, the stress score calculation apparatus 1 uses the notification controller 116 and the notification unit 140 to issue an alert. After execution of this step, the stress score calculation apparatus 1 ends the series of operations.

The stress score calculation apparatus 1 according to the fifth embodiment has been described hereinabove. According to the stress score calculation apparatus 1 according to the fifth embodiment, advantageous effects similar to those of the first embodiment, second embodiment, and third embodiment are obtained. In addition, the stress score calculation apparatus 1 according to the fifth embodiment issues an alert to the subject or the like in a case where the stress score 400 and the statistics 500 satisfy a predetermined criterion. By checking the alert, the subject is able to ascertain, for example, that they are experiencing excessive stress each day or are experiencing excessive stress due to a specific stress factor. As a result, it is expected that the subject will spontaneously change his/her living environment so as not to experience excessive stress. That is, the stress score calculation apparatus 1 is capable of prompting changes in the actions of the subject.

### OTHER EMBODIMENTS

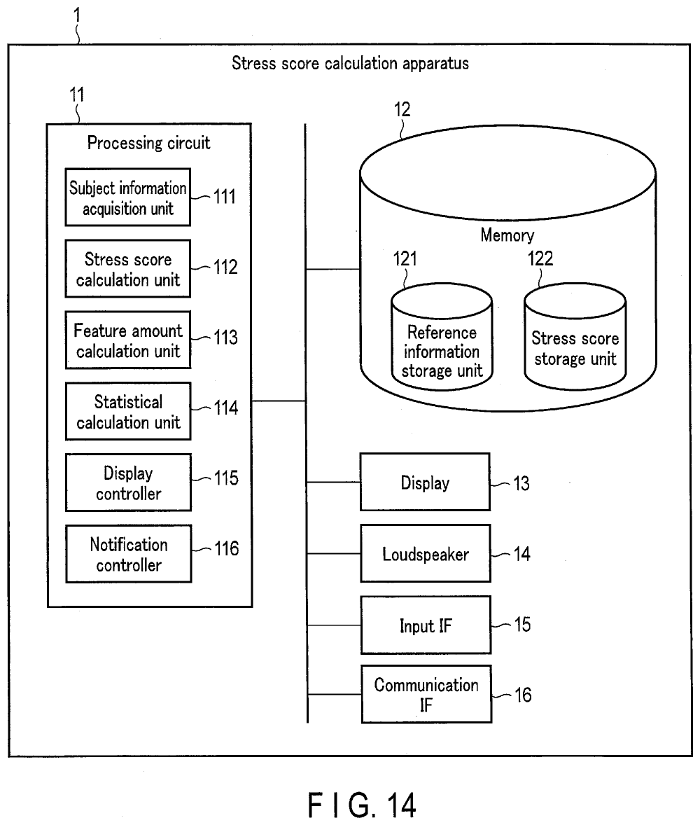

FIG. 14 is a block diagram illustrating a hardware configuration example of the stress score calculation apparatuses 1 according to the first to fifth embodiments. The stress score calculation apparatus 1 can be realized as an application that is installed on a portable terminal such as a smartphone. Alternatively, the stress score calculation apparatus 1 can be realized as various information processing apparatuses (examples: smartphone, wearable terminal, notebook PC, tablet terminal, desktop PC). The stress score calculation apparatus 1 includes a processing circuit 11, a memory 12, a display 13, a loudspeaker 14, an input IF 15, and a communication IF 16 as respective configurations. IF signifies interface. The respective configurations are communicably connected to each other by an internal bus. Note that each configuration may not be realized by individual hardware resources. For example, at least two of the configurations may be realized by the same hardware resource.

The processing circuit 11 controls the entire operation of the stress score calculation apparatus 1. The processing circuit 11 includes, as hardware resources, a processor such as a central processing unit (CPU), a micro processing unit (MPU), a graphics processing unit (CPU), a floating point unit (FPU), an application specific integrated circuit (ASIC), and a programmable logic device. For example, the processing circuit 11 realizes each unit (examples: the subject information acquisition unit 111, stress score calculation unit 112, feature amount calculation unit 113, statistic calculation unit 114, display controller 115, and notification controller 116) corresponding to each program by executing each program deployed in the memory 12 via the processor. Note that each unit can be realized by a processing circuit 11 which includes a single processor or a processing circuit 11 obtained by combining a plurality of processors. Note that the display controller 115 and the notification controller 116 may both be mounted on the processing circuit 11, or either one of the display controller 115 and the notification controller 116 may be mounted on the processing circuit 11.

The memory 12 stores information such as data and programs used by the processing circuit 11. The memory 12 has a semiconductor memory element such as a random access memory (RAM) as hardware. Note that the memory 12 may be a drive apparatus that reads and writes information from and to an external storage apparatus such as a magnetic disk (floppy (registered trademark) disk, hard disk), a magneto-optical disk (MO), an optical disk (CD, DVD, Blu-ray (registered trademark)), a flash memory (USB flash memory, memory card, SSD), or a magnetic tape. The storage area of the memory 12 may be inside the stress score calculation apparatus 1 or may be in an external storage apparatus. Note that the memory 12 may include at least one of the reference information storage unit 121 and stress score storage unit 122. The memory 12 is an example of a storage unit.

The display 13 displays information such as data generated by the processing circuit 11 and data stored in the memory 12. As the display 13, for example, a display such as a cathode ray tube (CRT) display, a liquid crystal display (LCD), a plasma display, an organic electroluminescence display (GELD: Organic Electro-Luminescence Display), or a tablet terminal can be used. The display 13 is an example of the display unit 130 and the notification unit 140.

The loudspeaker 14 issues an alert to the subject. The loudspeaker 14 is an example of the notification unit 140.

The input IF 15 receives an input from a user who uses the stress score calculation apparatus 1, converts the received input into an electric signal, and outputs the electric signal to the processing circuit 11. As the input IF 15, physical operation components such as a mouse, a keyboard, a trackball, a switch, a button, a joystick, a touch pad, a touch panel display, and a microphone can be used. Note that the input IF 15 may be an apparatus that receives an input from an external input apparatus which is separate from the stress score calculation apparatus 1, that converts the received input into an electrical signal, and that outputs the electrical signal to the processing circuit 11. When the user is the subject, the subject may manually input their own subject information 200 via the input IF 15. However, in a case where the user is an employee of a medical institution that is responsible for health management of the subject, the employee may manually input the subject information 200 of the subject via the input IF 15.

The communication IF 16 communicates various kinds of information between the stress score calculation apparatus 1 and an external apparatus. Any communication protocol can be used for the communication. For example, the stress score calculation apparatus 1 acquires the subject information 200 collected by the portable terminal owned by the subject via the communication IF 16. In addition, the stress score calculation apparatus 1 may transmit the stress score 400 to an external apparatus via the communication IF 16. The external apparatus may be a terminal owned by the subject.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A stress score calculation apparatus, comprising:
a memory that stores reference information which includes a reference value and/or a reference range pertaining to an action taken by a subject and/or a condition of an environment in which the subject is located that causes the subject an appropriate level of stress; and
a processing circuit configured to:
acquire subject information which includes at least one of action information pertaining to the action taken by the subject and environment information pertaining to the environment in which the subject is located; and
calculate a score pertaining to a degree of stress experienced by the subject, based on a comparison between the subject information and the reference information.

2. The stress score calculation apparatus according to claim 1,
wherein the action is sunbathing, exercise, a meal, use of an electronic device, a meeting, a nap, and/or a break, and
wherein the action information is a content, amount, acceleration, time, time zone, and a count and/or frequency of the action taken by the subject.

3. The stress score calculation apparatus according to claim 1,
wherein the environment information is a temperature, humidity, atmospheric pressure and/or solar radiation amount of the environment in which the subject is located.

4. The stress score calculation apparatus according to claim 1,
wherein the processing circuit calculates the score by adding a score indicating that a degree of stress experienced by the subject is appropriate in a case where the subject information satisfies the reference information, and adding a score indicating that a degree of stress experienced by the subject is not appropriate in a case where the subject information does not satisfy the reference information.

5. The stress score calculation apparatus according to claim 1,
wherein the processing circuit further acquires biometric information pertaining to bodily processes of the subject,
wherein the processing circuit calculates a feature amount pertaining to a stress reaction of the subject, based on the biometric information, and
wherein the processing circuit calculates the score also based on the feature amount.

6. The stress score calculation apparatus according to claim 5,
wherein the biometric information is a pulse wave, a pulse rate, a heart rate, an amount of sweating, an amount of urine, and/or an amount of saliva, and
wherein the feature amount is an LF/HF value, an LF value, an HF value, an ULF value, a VLF value, a total power value, an LF correction value, an HF correction value, an average HR value, an SDNN value, an RMSSD value, a CVRR value, an NN50 value, a pNN50 value, an αLF value, a μPA value, a CV-PI value, a Lorenz plot, and/or a recurrent plot.

7. The stress score calculation apparatus according to claim 5,
wherein the processing circuit calculates the score by adding a score indicating that a degree of stress experienced by the subject is appropriate in a case where the feature amount is less than or equal to a threshold value or lies within a predetermined range, and adding a score indicating that a degree of stress experienced by the subject is not appropriate in a case where the feature amount exceeds the threshold value or lies outside the predetermined range.

8. The stress score calculation apparatus according to claim 1,
wherein the memory stores the score each time the score is calculated, and
wherein the processing circuit calculates a statistic pertaining to each of the stored scores.

9. The stress score calculation apparatus according to claim 8,
wherein the statistic is an average value, a total value, a minimum value, a maximum value, a median value, a deviation value, a mode value, a range, a variance, a standard deviation, a skewness, and/or kurtosis.

10. The stress score calculation apparatus according to claim 8,
wherein the processing circuit performs control to display the stored scores and/or the stored statistics on a display.

11. The stress score calculation apparatus according to claim 10,
wherein the processing circuit performs control to cause the display to display the scores stored in a first predetermined period and an average value and/or a total value of the scores, and the scores stored in a second predetermined period included in the first predetermined period and an average value and/or a total value of the scores.

12. The stress score calculation apparatus according to claim 8,
wherein the processing circuit performs control to cause a display and/or a loudspeaker to issue an alert in a case where the stored scores and/or the stored statistics satisfy a predetermined criterion.

13. A stress score calculation method, comprising:
storing reference information which includes a reference value and/or a reference range pertaining to an action taken by a subject and/or a condition of an environment in which the subject is located that causes the subject an appropriate level of stress,
acquiring subject information which includes at least one of action information pertaining to the action taken by the subject and environment information pertaining to the environment in which the subject is located, and
calculating a score pertaining to a degree of stress experienced by the subject, based on a comparison between the subject information and the reference information.

14. A non-transitory computer readable medium including computer executable instructions, wherein the instructions, when executed by a processor, cause the processor to perform a method comprising:
storing reference information which includes a reference value and/or a reference range pertaining to an action taken by a subject and/or a condition of an environment in which the subject is located that causes the subject an appropriate level of stress,
acquiring subject information which includes at least one of action information pertaining to the action taken by the subject and environment information pertaining to the environment in which the subject is located, and
calculating a score pertaining to a degree of stress experienced by the subject, based on a comparison between the subject information and the reference information.
